(19) United States
(12) Patent Application Publication
(10) Pub. No.: US 2012/0303019 A1
(43) Pub. Date: Nov. 29, 2012

(54) Kind of cardiac ablation catheter with guide-wire

(76) Inventors: Xue Zhao, Shanghai (CN); Xiabing Zhao, Shanghai (CN)

(21) Appl. No.: 13/304,329

(22) Filed: Nov. 24, 2011

Related U.S. Application Data
(60) Provisional application No. 61/427,176, filed on Dec. 25, 2010.

(51) Int. Cl. A61B 18/12

(52) U.S. Cl. 606/41

(57) ABSTRACT

Existing ordinary cardiac ablation catheter, especially, the catheter for atrial fibrillation ablation is easily skipped off the target site of ablation when it is being moved from one site to another. The present invention provides a cardiac ablation catheter with a guide-wire. During a procedure of pulmonary vein electrical isolation, the guide-wire needs to be advanced into the distal end of pulmonary vein. With the assistant of the powerful sustainability from the guide-wire, the ablation catheter will be more controllable.

### THE TECHNICAL FIELD

This invention relates to a cardiac ablation catheter for therapy of arrhythmia by radiofrequency ablation, especially for use in atrial fibrillation ablation.

### BACKGROUND TECHNOLOGY

At present, the known ablation catheter for radiofrequency catheter ablation of cardiac arrhythmias needs to be moved accurately and to have a reliable and stable contact with ablation site (the lesion) during the procedure of ablation. The catheter can be moved in cardiac chamber not only by direct pushing, drawing or rotating the joystick on the tail end of the catheter, but also by adjusting the curvature of the distal part of the catheter so as to reach and contact with the target site. One of the important functional characteristics of ablation catheter is that the curvature of distal part of the catheter can be adjusted via the joystick on the proximal part of the catheter, which is the critical mode of operation for ablation catheter to reach and contact against the target site so that radiofrequency current can be delivered through the distal electrodes. Ablation target site for supraventricular tachycardia is usually one point; an experienced doctor can manipulate the ablation catheter to reach and contact the target site in a short time. The complexity of the pathogenesis in atrial fibrillation leads to the particularity of atrial fibrillation ablation. The target of atrial fibrillation ablation is not just one point; the basic strategy for atrial fibrillation ablation is pulmonary vein electric isolation by circumferential pulmonary vein ablation. Ablation site of paroxysmal atrial fibrillation is located at the pulmonary vein antrum in the left atrium. The circular linear ablation must be continuous without gap because ablation gap means ablation failure. Moreover, the ablation line should keep 1 cm away from the pulmonary vein orifice, otherwise it may cause permanent pulmonary vein stenosis. It is necessary for ablation of chronic atrial fibrillation to make additional linear ablations except for pulmonary vein electrical isolation. The key skill for successful ablation of atrial fibrillation is to control ablation catheter finely so that the distal electrode of the ablation catheter can have a reliable and stable contact with the target site. The operation distance of atrial fibrillation ablation is relatively far because the ablation catheter is inserted from femoral vein and needs to get into the left atrium via atrial septal puncture, so the doctor has to manipulate the catheter from outside of the body. Patients with atrial fibrillation usually suffer from an expansion of the left atrial volume at different degrees, and the longer atrial fibrillation lasts, the more severe structural heart disease will be, and the larger left atrium is; The anatomic structure of pulmonary vein antrum is extremely irregular, and much variable among individuals; Heart is an organ with continuous beating, the heart beating during atrial fibrillation is completely irregular, breathing movement also significantly affects the spatial location of heart. Based on the above reasons, it is understandable that the doctor must be very skillful so that the procedure of atrial fibrillation ablation can be completed successfully. It is difficult to position an ablation catheter accurately onto a specific site in left atrium, and it is much more difficult to keep the tip of the catheter moving round and make a circumferential linear ablation in pulmonary vein antrum; The ablation line is made of a number of ablation points, and the ablation line is required to be complete and continuous without gap between ablation points. This shows that pulmonary vein isolation is indeed a high-skill work. It has been shown by clinical practice that even if circumferential pulmonary vein ablation is finished, it is still hard to guarantee that pulmonary vein is electrically isolated from the left atrium. Even an experienced electrophysiological doctor still feels difficulty to move the ablation catheter towards an optimal direction and optimal distance, and to make the catheter contacting with the left atrial endocardium accurately. It occurs frequently for the ablation catheter to skip off the ablation point; Poor contact and skip-off of the catheter will directly affect the efficiency and success of the ablation procedure. If the ablation catheter hops off the ablation site, the doctor must take time to reset it back, then X-ray exposure and operating time will increase, furthermore, discontinuities and imperfection of the circumferential ablation line will be resulted in too. This will reduce success rate of atrial fibrillation ablation and also increase potential recurrences of atrial fibrillation.

A cardiac ablation catheter can be divided into three parts according to its surface structure and functions, namely, the distal part of the catheter (tip), the body part of the catheter and the proximal part of the catheter (joystick at the tail end). The distal part of the catheter is a part, in which the curvature can be adjusted by the joystick, mainly including four electrodes, thermocouple head, outlet of cool saline and so on. All the electrodes are made of platinum or platinum iridium alloy, in which each electrode is connected with copper wire, go through the body of the catheter to the tail end of the catheter. The body of the catheter is the part between the distal part and the joystick of the catheter, which occupies most of the length of the catheter and capsulate electrode wires, saline perfusion tube, control wire, etc. The distal part and the body part of the catheter are covered with polyurethane or nylon polymer material. The proximal part of the catheter is composed of a joystick, by which the curvature of the distal part of the catheter can be controlled. In addition, there are a number of electrode circuit interfaces and an inlet of cool saline in joystick. The tail end of the catheter will be connected by a cable with an electrophysiological recording device and radiofrequency delivering machine during procedure.

An ablation catheter, made in USA with a brand of ThermoCool, Biosense Webster has been widely used clinically in atrial fibrillation ablation. The inherent structure on the distal end of the catheter includes four electrodes, temperature sensor, outlet of cool saline, magnetic inductor and so on. In order to improve the controllability and contact stability of ablation catheter in the left atrium during procedure, large-scale permeance equipment or manual robot operating system are currently produced for clinical application. But because they are extremely expensive with a substantial increase in cost of ablation procedure, these equipments are too difficult to be widely used in hospitals. Medtronic, Inc. USA is studying an ablation catheter of atrial fibrillation named of “Frontier”, which is a circular electrode ablation catheter based on an adaptation of a circular mapping electrode catheter. This catheter was designed to use for circumferential pulmonary vein ablation. It can improve the contact of catheter with endocardium and realize current delivery simultaneously with multi-electrodes. If a linear ablation is necessary on the roof of left atrium or within the coronary vein sinus, the Frontier catheter would be useless and must be replaced, which undoubtedly increase the cost of ablation procedure. An endoscopic ablation catheter was recently reported for atrial fibrillation ablation, which can carry out circumferential pulmonary vein ablation under direct vision. However, as a disposable device, it will consume a cardiovascular endoscope at each procedure, and undoubtedly increase the cost of ablation procedure. There is an ablation catheter designed in China Patent (200610063117.X) with a coaxial central hole inside the catheter. After a circular guide-wire is inserted via the central hole to the distal tip of the catheter, the distal part of the catheter will be curved to become a circle, so circumferential pulmonary vein ablation can be finished at one time of radiofrequency deliver. However, this ablation catheter may produce more ablation gaps due to unreliable contact with tissue. Furthermore, if a linear ablation is necessary on the roof of left atrium or within the coronary vein sinus, this catheter would be useless and one more ordinary ablation catheter must be use.

### INVENTION

The procedure of pulmonary vein electrical isolation can be described simply to draw a circle in both pulmonary vein antria respectively by moving the ablation catheter. Basically, it is the simplest and most accurate method to draw a circle by using a compass. Similarly, as long as adding a guide-wire on the cardiac ablation catheter, which can play a supporting role of central axis or as the fulcrum, it will be easy to draw a circle by using the distal electrode of ablation catheter. Currently, as to the available cardiac ablation catheter, especially the catheter for atrial fibrillation ablation, the moving direction and distance of it in the heart chamber cannot be controlled as effectively as we expect, and also the ablation catheter cannot contact very well to the surface of the atrial endocardium. In order to overcome these defects, the present invention provides an ablation catheter with a guide-wire. The guide-wire can be inserted into the catheter from the proximal to the body part of the catheter, or from the joystick at the tail end of the catheter. The ablation catheter containing a guide-wire will be inserted into the left atrium during the procedure. The guide-wire can be pushed out from the distal hole which is located just on the interface between the body part and distal part of the catheter.

While the guide-wire is advanced into the distal end of the pulmonary vein, the ablation catheter will be fixed to the space of the pulmonary vein antrum. Thus, the distal end of the ablation catheter would be rotated ad libitum and stably around the guide-wire. The rotating radius of the distal electrode of the catheter will be determined by the curvature size of the distal part of the catheter, which is usually flexible. The movement space of the catheter is completely restricted to the pulmonary vein antrum. Supported by the guide-wire, therefore, the direction and distance of the catheter movement can be effectively controlled and the catheter can also contact very well with the endocardium. Because of synchronous movement of the guide-wire with respiratory rhythm, the guide-wire will buffer the influence of breath on the catheter location and increase the contacting stability of the catheter with atrial endocardium.

In order to solve the technology problems existed in practice of atrial fibrillation ablation by using the routine cardiac ablation catheter, the present invention adopts a technology solution: the invention of a cardiac ablation catheter with a guide-wire. The way to realize is to set a guide-wire cavity within the body part of the cardiac ablation catheter, or within the body part and the joystick of the routine cardiac ablation catheter. The guide-wire is going to be inserted into the guide-wire cavity in the catheter during procedure. The guide-wire cavity is set at the lateral side of the body part of the catheter, being the different axis with the outer edge of the body part of the catheter.

The cross-section of the guide-wire cavity shows arch or circular-shape.

The cross-section structure of the body part of the catheter shows circular shape. The distal hole of the guide-wire cavity is just set at the interface between the distal part and body part of the catheter. The proximal hole of the guide-wire cavity is set at the proximal to the body part of the catheter near the joystick, or at the tail end of the joystick. The guide-wire cavity starts at its proximal hole and ends at its distal hole. The wall of the guide-wire cavity at the distal hole makes a smooth transition and becomes a streamline with the surface of the distal part of the catheter, which will reduce the resistance when the catheter is pushed forward. The proximal hole of the guide-wire cavity connects with a segment of soft tube, on which there is a valve or tightening device to prevent the outflow of blood and the slide out of the guide-wire. On the soft tube, there is a side hole too, which will be connected to a saline infusion set for flushing of the guide-wire cavity or for drug administration. In clinical application, the guide-wire will be inserted from the tail hole of the soft tube, pushed into the guide-wire cavity, comes out from the distal hole of the guide-wire cavity, and then reaches the blood vessel or cardiac chamber.

The cross-section of the guide-wire shows arch or circular shape. The distal end of the guide-wire is shaped as the letter “J”, which is soft and flexible. The mould of the guide-wire is the same as that of the guide-wire cavity, and both sizes are matched properly with each other. The guide-wire can go smoothly within the guide-wire cavity with a minimum gap between guide-wire and the guide-wire cavity. The guide-wire is much longer than the length of the guide-wire cavity. The guide-wire has a certain rigidity and supporting-force against the catheter.

The guide-wire with the arch-shaped cross section helps to increase the level of integration among the guide-wire and other structures of the catheter, reducing the cross-sectional area of the whole catheter, increasing the contact area with tissue when it is in the pulmonary vein. And the guide-wire with the circular-shaped cross section also helps flexible rotation between the catheter and the guide-wire.

The beneficial effect of the invention is that, during circumferential pulmonary vein ablation, the ablation catheter can be limited to the pulmonary vein antrum, and the moving direction and distance of the ablation catheter can be controlled accurately. Furthermore, it can prevent effectively the ablation catheter from skipping off the ablation target region while a directional movement is needed. After the guide-wire is withdrawn to the guide-wire cavity, the present invention can also be used for ablation at the other areas, such as linear ablation in both atria. So this catheter keeps all the functions of a routine catheter for atrial fibrillation ablation. Because of the enhanced contact stability of the ablation catheter with the atrial wall, the ablation efficiency will be improved greatly, operation time reduced and ultimately the success rate of atrial fibrillation ablation improved.

### DESCRIPTION OF THE DRAWINGS

Further description combined with drawings and embodiments of the present invention as below.

FIG. 1 is a structural schematic drawing showing the cardiac ablation catheter through this invention.

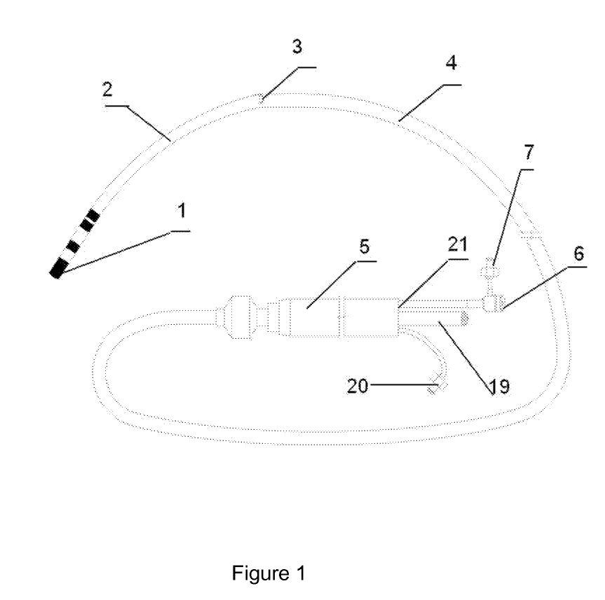

FIG. 2 is a structural schematic drawing showing the guide-wire and the cross-section of the guide-wire.

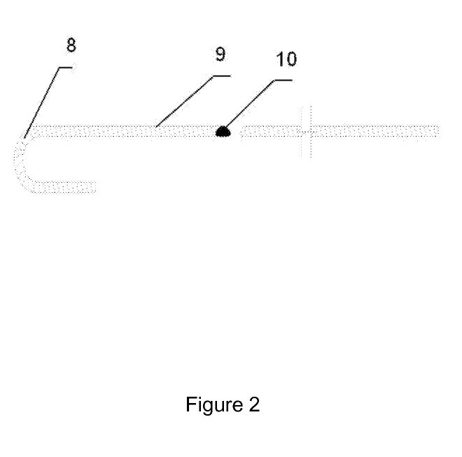

FIG. 3 is a partial structural schematic drawing showing a region of the distal hole of the guide-wire cavity in the cardiac ablation catheter with the guide-wire.

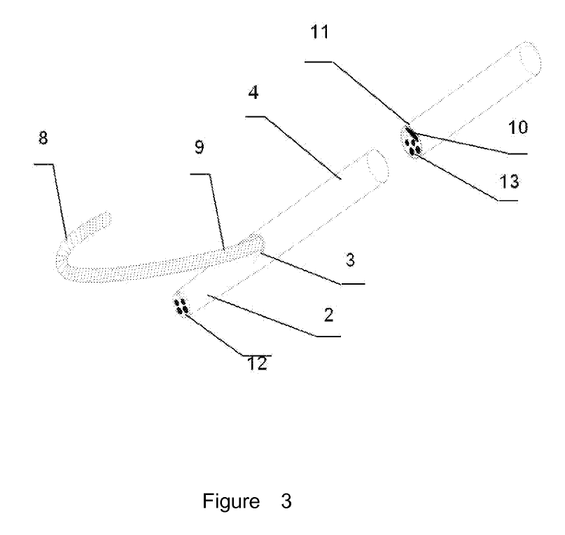

FIG. 4 is an embodiment diagram showing the cardiac ablation catheter with the guide-wire.

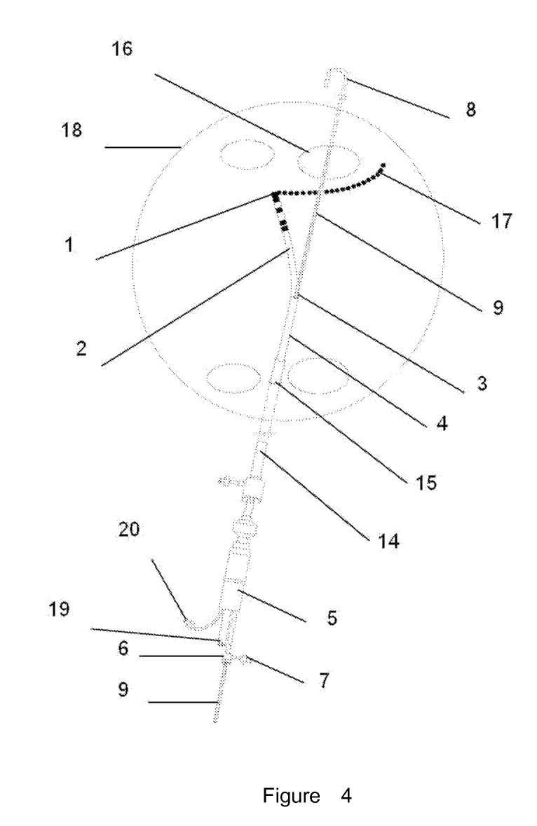

In these drawings, 1. distal electrode of the catheter, 2. the distal part of the catheter, 3. the distal hole of the guide-wire cavity, 4. the body part of the catheter, 5. the joystick in the tail end of the catheter, 6. the tail hole of the soft tube, 7. the side hole of the soft tube, 8. the distal end of the guide-wire, 9. the guide-wire, 10. the cross-section of the guide-wire, 11. the cross-section of the guide-wire cavity, 12. the cross-section of the distal part of the catheter, 13. the cross-section of the body part of the catheter, 14. the catheter sheath for atrial septal puncture, 15. the puncture aperturae of the atrial septum, 16. pulmonary vein orifice, 17. ablation line, 18. left atrium, 19. the cable at the tail end of the catheter, 20. the influent hole of cold saline infusion, 21. the tail hole of the guide-wire cavity.

### DETAILED DESCRIPTION OF THE EMBODIMENTS

At the beginning of the procedure for atrial fibrillation ablation, a catheter sheath for atrial septal puncture 14, is introduced into the left atrium 18, via the puncture aperturae of the atrial septum 15. And then the cardiac ablation catheter in the present invention will be applied. At first, the distal end of the guide-wire 8, is being inserted into the guide-wire cavity from the tail hole of the soft tube 6, making sure that the distal end of the guide-wire 8, should not emerge from the distal hole of the guide-wire cavity 3, at this time. The cardiac ablation catheter with guide-wire 9, is introduced through the catheter sheath 14, into the left atrium 18, continually advanced to the pulmonary vein orifice 16. After the joystick of the catheter 5, is fixed temporally, the guide-wire 9, needs to be pushed forward, and the distal end of the guide-wire 8 is made to come out of the distal hole of the guide-wire cavity 3. The distal end of the guide-wire 8 needs to be sent optimally to the distal end of the pulmonary vein. Being monitored under the X-ray, and/or three-dimensional atrial mapping system, the distal electrode of the catheter 1, is being positioned in the pulmonary vein antrum; After the guide-wire is fixed at the tail hole of the soft tube 6, by the tightening device or valve, the distal end of the guide-wire 8, is kept in the distal end of the pulmonary vein. In addition, the body part of the catheter 4, the guide-wire 9, and the catheter sheath 14, all together are being fixed by the puncture aperturae of the atrial septum 15. Thus, the guide-wire 9, can play a role of rotational axis for the distal electrode of the catheter 1. The ablation catheter can be rotated clockwise or counterclockwise, thus the distal electrode of the catheter 1, can be moved around the pulmonary vein orifice 16, in a more convenient, stable, fast and accurate way, and doctors need no longer to worry about the catheter skipping off the ablation target region while the catheter needs moving. The stable contacting of the distal electrode of the catheter 1, with endocardium in pulmonary vein antrum can be easily maintained by pushing forward the catheter. The radius of circumferential pulmonary vein ablation can also be determined by just adjusting curvature of the distal part of the catheter 2. The ablation line 17, is on behalf of a trajectory of the distal electrode of the catheter at radiofrequency delivering. When the distal end of the guide-wire 8, is pulled back to the guide-wire cavity, the ablation catheter can be used as an ordinary ablation catheter for linear ablation in other parts of the atria, which is usually necessary during the procedure of atrial fibrillation ablation.

1. A kind of cardiac ablation catheter, which is clinically used for treatment of cardiac arrhythmia, including distal part of the catheter (2), body part of the catheter (4) and joystick (5) at the tail end of the catheter, which is characterized by: there is a guide-wire cavity inside said body part of the catheter (4), or inside said body part of the catheter (4) and the joystick (5) at the tail end of the catheter, and a guide-wire (9) which matches the guide-wire cavity.

2. The catheter according to claim 1, wherein the distal hole (3) of the guide-wire cavity is set at the interface between said distal part of the catheter (2) and said the body part of the catheter (4).

3. The catheter according to claim 1, wherein the proximal hole (21) of the guide-wire cavity is set at the proximal to the body part (4) of the catheter near the joystick (5), or at the tail end of the joystick (5).

4. The catheter according to claim 1, wherein the guide-wire cavity is set at the lateral side of the body part of the catheter (4), being the different axis with the outer edge of said the body part of the catheter (4).

5. The catheter according to claim 1, wherein the cross-section of the guide-wire cavity (11) shows arch or circular-shape.

6. The catheter according to claim 1, wherein cross-section of the guide-wire (10) shows arch or circular-shape, distal end of the guide-wire (8) is soft and flexible with curved-shape.

7. The catheter according to claim 1, wherein the guide-wire (9) is longer than the length of the guide-wire cavity.

8. The catheter according to claim 2, wherein both peritreme of distal hole of the guide-wire cavity (3) and surface of distal part of the catheter (2) move smoothly with streamlined.

9. The catheter according to claim 2, wherein the proximal hole (21) of the guide-wire cavity connects with a segment of soft tube, on which there is a valve or tightening device and a side hole (7).

10. The catheter according to claims 3 to 4, wherein the mould of the guide-wire (10) is the same as that of the guide-wire cavity (11) with matching size.
